(19) United States
(12) Patent Application Publication
(10) Pub. No.: US 2021/0213633 A1
(43) Pub. Date: Jul. 15, 2021

(54) Knife Handle and Cover

(71) Applicant: Surgistar, Inc., Vista, CA (US)

(72) Inventor: Neil Chaudhary, Vista, CA (US)

(21) Appl. No.: 16/739,828

(22) Filed: Jan. 10, 2020

(51) Int. Cl. B26B 29/02; A61B 17/3211

(52) U.S. Cl. CPC B26B 29/025; A61B 17/3211

(57) ABSTRACT

A knife cover and handle assembly includes a cover having a housing with a retaining area that couples to a handle of a knife. The cover is configured to prevent movement of the blade or handle of a knife within the housing of the cover, without using foam that could dull the blade. The cover can be replaceable, disposable, and/or reusable. The knife cover and handle assembly reduces manufacturing time and cost, and environmental impact.

### BACKGROUND

Knives, such as surgical, knives have sharp edges that need to be secured during shipping and handling such as to protect the blade of the knife from damage, and to protect users and/or shipping carriers from injury. Additionally, the packaging of the knife requires protection from damage by the blade of the knife such as to maintain packaging integrity and/or sterility. This has historically been achieved by a blade protector. Common methods of securing the sharp edges of the blade include plastic trays or foam devices that secure the entire knife. Foam securing methods involve insertion of the blade into the foam, thereby cutting the foam to allow the blade to remain situated within the foam. This cutting of the foam means the blade has been used, and potentially dulled, before ever being used in surgery. Because plastics, foams, and similar polymers can include excessive amounts of material and can have environmental impacts upon disposal, there is a need for packaging that reduces waste and manufacturing costs while effectively protecting the blade of a knife.

### BRIEF SUMMARY

Described herein is a knife handle and cover assembly, and methods of manufacture thereof, that covers only a blade and a portion of a handle of a knife, while fully restraining the knife and protecting the blade, and not subjecting the blade to cutting foam prior to use. Aspects of the current subject matter related to a system and method for securing a knife blade and handle.

In an aspect, a knife cover and handle assembly is provided. The cover includes a housing. The housing includes a retaining area, a retention feature, and a coupling mechanism. The coupling mechanism is configured to receive a handle. The handle includes a body and a blade. The body includes a proximal end, a distal end, a neck, and a coupling feature. The cover is configured to retain the handle such that the handle and blade do not move relative to the cover.

In some embodiments, the coupling mechanism is a recess. In some embodiments, the coupling feature is a detent. In some embodiments, the blade is a surgical blade

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### BRIEF DESCRIPTION OF THE DRAWINGS

FIG. 1 illustrates an embodiment of a knife handle coupled to a cover, consistent with implementations of the current subject matter.

FIG. 2 is a side perspective, enlarged view of the knife handle and cover of FIG. 1.

FIG. 3 is a side plan, enlarged view of the knife handle and cover of FIG. 1.

FIG. 4 is top plan, enlarged view of the knife handle and cover of FIG. 1.

FIG. 5 is a to perspective, enlarged view of the knife handle and cover of FIG. 1.

FIG. 6 is a side perspective view of the knife handle of FIG. 1 uncoupled from the cover of FIG. 1.

FIG. 7 is a side perspective, enlarged view of an embodiment of a coupling mechanism for the knife handle and cover of FIG. 1, consistent with implementations of the current subject matter.

FIG. 8 is a bottom perspective, enlarged view of the coupling mechanism of FIG. 6.

### DETAILED DESCRIPTION

Disclosed herein is a knife cover with a housing configured to retain a handle and a blade of a knife, such as a surgical knife. For example, the knife cover can have a housing with a retaining area, a retention feature, and a coupling mechanism. In some embodiments, the coupling mechanism can be configured to receive a handle. The handle can include a body with a proximal end and a distal end, a tapered neck at the distal end of the body, a coupling feature at the distal end of the body and proximal to the neck, and a blade attached to the neck and extending outwardly therefrom. In some embodiments, the cover can be configured to retain the handle such that the blade cannot puncture, pierce, or otherwise compromise the structure and/or sterility of the cover.

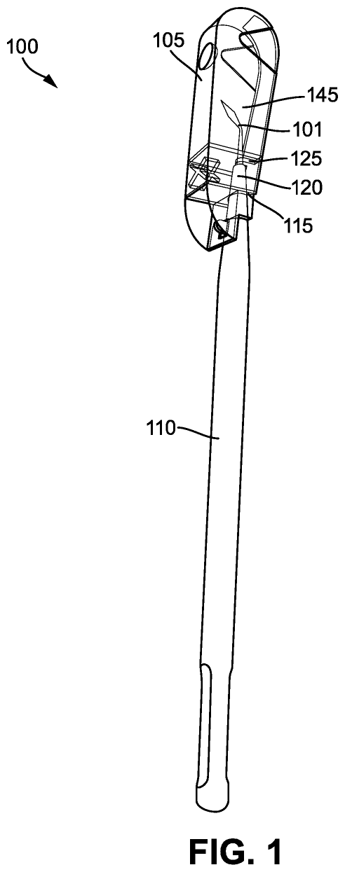

FIG. 1 illustrates an embodiment of a knife handle and cover assembly 100, consistent with implementations of the current subject matter. In some embodiments, the knife handle and cover assembly 100 can include a cover 105 and a handle 110. For example, the handle 110 can include a neck 120 and can be coupled to a blade 101 such as at a distal end of the handle 110. The cover 105 is an outer structure that defines an internal cavity that is sized and shaped to receive and enclose the blade 101 and optionally at least a portion of the handle 110. The shape of the cover 105 can vary and is not limited to the shape shown in the figures.

In some embodiments, the cover 105 can include a retention area 145, a retention feature 125, and a cradle 115. For example, the retention area 145 can be a cavity or enclosure configured to receive the blade 101. The retention feature 125 can be, for example, a structure configured to receive or otherwise couple to the neck 120 such as at a point where the neck 120 meets the blade 101. The cradle 115 can be a structure configured to receive the neck 120 at a point where the neck 120 meets the handle 110. For example, the cradle 115 can include two opposing sides and a depression therebetween, the depression configured to have an approximately complementary shape to the neck 120 and the handle 110, such that the neck 120 rests in the depression of the cradle 115. In some embodiments, the cover 105 can be formed from any one of: polycarbonate, polystyrene, acrylonitrile butadiene styrene (ABS), polysulfone, or a combination thereof. It should be appreciated that the cover 105 can be formed from other suitable polymers, plastics, or foams without departing from the scope of this disclosure. In some embodiments, the cover 105 may be formed of a material capable of withstanding increased temperatures and pressures, for example those temperatures and pressures required for sterilization, such as in an autoclave.

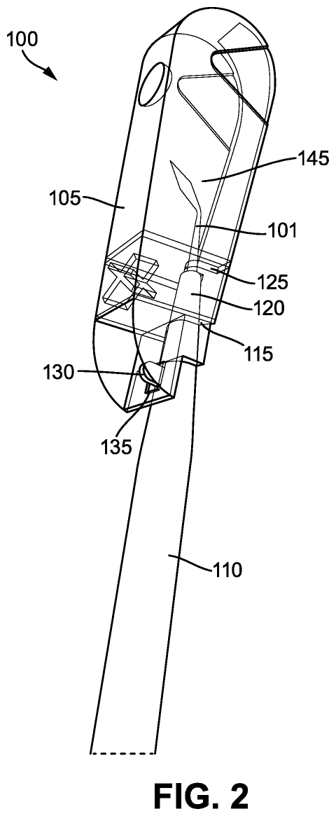

As shown in FIG. 2, the knife handle and cover assembly 100 can further include a coupling mechanism. For example, the handle 110 can include a detent 130 (for example, a dimple, protrusion, tab, etc.) protruding outwardly from the handle, and the cover 105 can include a corresponding recess 135 that can fit and/or otherwise snap over the detent 130 such that the detent 130 and the recess 135 engage one another. In some embodiments, the recess 135 is a depression or aperture, such as a slit. Such a coupling mechanism can provide enough support to hold the handle 110 in place and ensure good contact between the detent 130 and the recess 135, while allowing release of the detent 130 from the recess 135 when a user pulls with reasonable force on the handle 110 to disengage the handle 110 from the cover 105. For example, the cover 105 may be removable and replaceable, such as to protect the blade 101 during disposal.

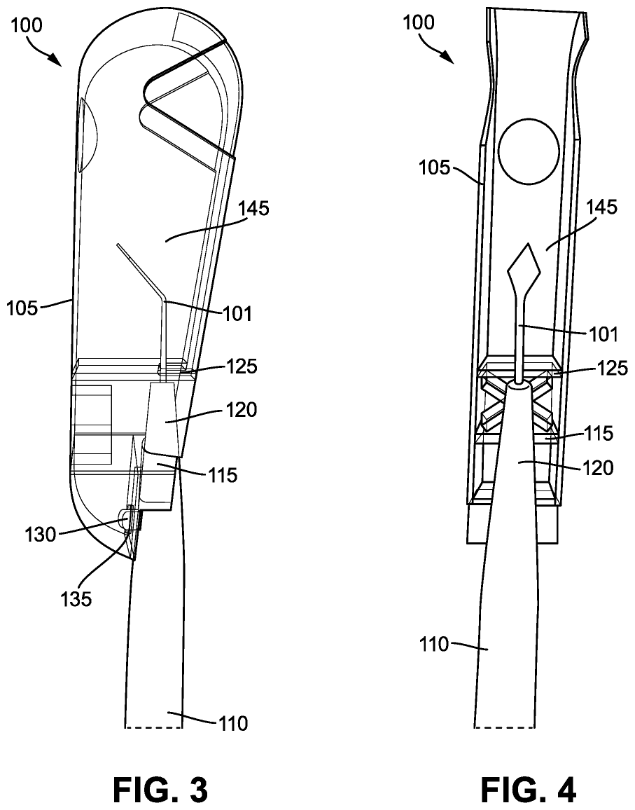

FIG. 3 is a side view of the knife handle and cover assembly 100. In some embodiments, the cover 105 can receive the handle 110 and blade 101, such that the detent 130 is held in place in the recess 135, and the neck 120 is held in place by the cradle 115 and the retention feature 125. For example, the cover 105 can be configured to receive the handle 110 and blade 101 such that there is no freedom of movement between the cover 105 and the handle 110 and blade 101. As shown in FIG. 3, the retention area 145 can be configured to receive a curved blade such as the blade 101, however it should be appreciated that the retention area 145 can receive a variety of blade shapes, sizes, and configurations without departing from the scope of this disclosure.

FIG. 4 is a top view of the knife handle and cover assembly 100. As shown in FIGS. 3-4, if the blade 101 is curved toward a plane, the detent 130 can be formed on a portion of the handle 110 that is in the same plane that the blade 101 curves toward, such that the sharp edges of the blade 101 extends into the retention area 145 and away from the open top of the cover 105. Such an orientation can prevent the sharp edges of a curved blade 101 from extending outwardly from the open top of the cover 105.

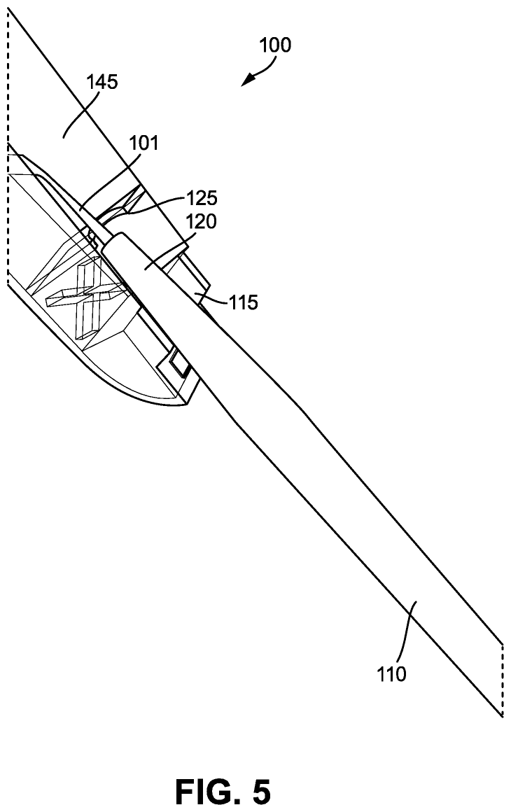

As shown in FIG. 5, the cover 105 can be configured such that it covers only the blade 101 and the neck 120 of a knife. Such a reduction in size compared to traditional knife protectors can provide a significant reduction in manufacturing time and costs. Additionally, the reduction in size reduces the environmental impact of the cover 105. Furthermore, because the cover 105 can include a releasable coupling mechanism, the cover 105 may be replaced onto a used knife for sharps disposal.

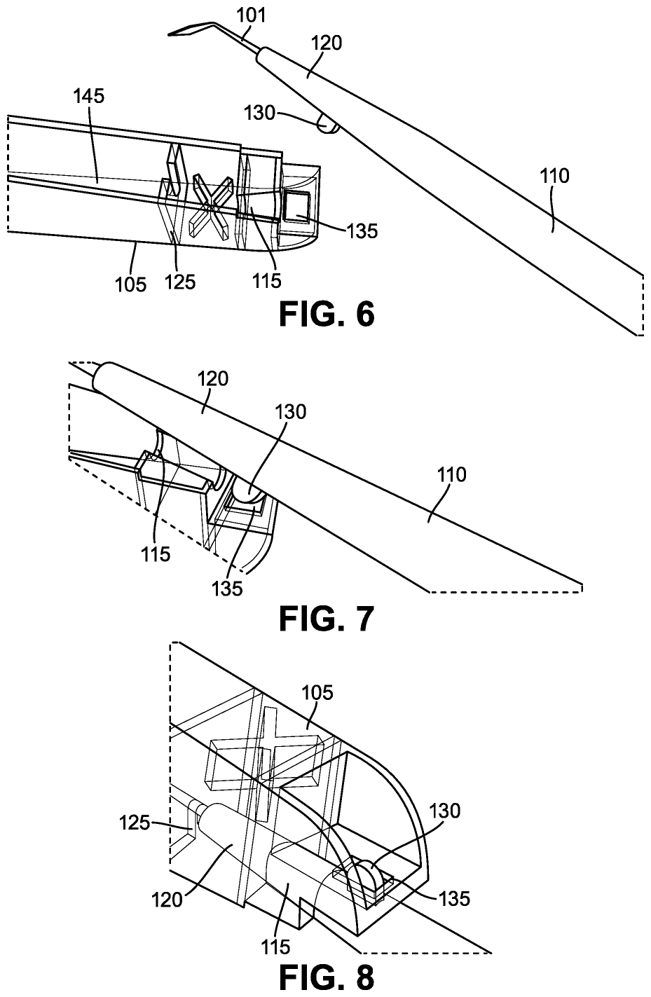

FIGS. 6-8 illustrate steps of coupling the cover 105 to the knife via the detent 130 and recess 135. FIG. 6 shows the cover 105 uncoupled from the handle 110 and blade 101. The detent 130 can be seen on the handle 110, and is positioned such that the blade 101 will curve downwardly into the retention area 145 when placed into the cover 105. FIG. 7 illustrates the detent 130 being inserted into the recess 135.

FIG. 8 is a bottom view of the neck 120 and detent 130 coupled to the cover 105. As shown in FIG. 8, the neck 120 can be held in place by the cradle 115 and the retention feature 125. For example, the cover 105 can be configured to be in contact with the neck 120 and the detent 130, without contacting the entirety of the handle 110. It should be appreciated, however, that the cover 105 can cover a portion of the handle 110 without departing from the scope of this disclosure.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A knife cover and handle assembly, the cover comprising:
a housing, the housing comprising:
a retaining area,
a retention feature, and
a coupling mechanism;
wherein the coupling mechanism is configured to receive a handle, the handle comprising:
a body, the body comprising:
a proximal end,
a distal end, and
a neck, and
a coupling feature; and
a blade;
wherein the cover is configured to retain the handle such that the handle and blade do not move relative to the cover.

2. The knife cover and handle assembly of claim 1, wherein the coupling mechanism is a recess.

3. The knife cover and handle assembly of claim 1, wherein the coupling feature is a detent.

4. The knife cover and handle assembly of claim 1, wherein the blade is a surgical blade.
